(19) United States
(12) Patent Application Publication
(10) Pub. No.: US 2026/0209835 A1
(43) Pub. Date: Jul. 23, 2026

(54) METHOD OF NUCLEIC ACID QUANTIFICATION USED TO INCREASE DYNAMIC RANGE IN DIGITAL PCR

(71) Applicant: QIAGEN GmbH, Hilden (DE)

(72) Inventors: Miroslav VRANES, Hilden (DE); Stefan Otto CORNELIUS, Hilden (DE); Mario SCHERER, Hilden (DE); Ralf PEIST, Hilden (DE)

(21) Appl. No.: 19/140,643

(22) PCT Filed: Dec. 20, 2023
(86) PCT No.: PCT/EP2023/086981

§ 371 Date: Jun. 18, 2025

(30) Foreign Application Priority Data
Dec. 23, 2022 (EP) 22216408.9
Jan. 19, 2023 (EP) 23152530.4

(51) Int. Cl. C12Q 1/6851; C12Q 1/6806; C12Q 1/686; C12Q 1/6876

(52) U.S. Cl. CPC C12Q 1/6851; C12Q 1/6806; C12Q 1/686; C12Q 1/6876

(57) ABSTRACT

The present invention relates to a method of nucleic acid quantification, the method comprising the steps of (i) providing a sample comprising a plurality of nucleic acid molecules; (ii) amplifying at least two loci within said plurality of nucleic acid molecules, wherein the at least two loci comprise at least one multicopy locus and at least one single copy locus and wherein the amplification method is a digital polymerase chain reaction method; (iii) detecting the at least two amplification products; and (iv) quantifying the amount of the plurality of nucleic acid molecules present in the sample; wherein the quantifiable nucleic acid concentration ranges for the at least one multicopy locus and the at least one single copy locus are overlapping. The invention also relates to a kit for use in the methods according to the invention.

### FIELD OF INVENTION

The present invention is in the field of molecular biology, in particular in the field of analytical and forensic sciences. Furthermore, the invention is in the field of nucleic acid amplification and quantification, more specifically in the field of nucleic acid quantification over a broad range of DNA concentrations in digital PCR.

### BACKGROUND

DNA-based diagnostic and analyses methods are of rising importance in many areas, for example in the fields of diagnostic-, genetic-, forensic-, and food testing or for the detection of genetically modified organisms (GMO). The more accurate the determination of the amount of (amplifiable) DNA, the more successful the subsequent analyses and diagnostic methods.

Currently, quantitative real-time PCR (qPCR) is a widely used method for DNA or RNA quantification. As reviewed in Quan et al. (Quan et al., 2018, MDPI, “dPCR: A Technology Review”), quantitative real-time PCR is based on conventional PCR. In conventional PCR, target DNA is amplified in multiple cycles, wherein at the end of each of a 100% efficient PCR cycle the number of target DNA molecules is doubled (exponential amplification). Hence, 2ⁿ copies can theoretically be produced after n cycles. However, in practice, the PCR reagents will be depleted at one point and amplification products will self-anneal, resulting in reduced amplification efficiency until a plateau is reached and the amplification process saturates. At the end of a conventional PCR reaction, the amplified products can be analyzed using agarose gel electrophoresis (end-point measurement). The specificity of a conventional PCR relies on sequence hybridization. The sensitivity of a conventional PCR depends on enzyme-based amplification (Quan et al., 2018, MDPI, “dPCR: A Technology Review”).

As mentioned above, quantitative real-time PCR is based on conventional PCR, but the amount of amplified PCR products are measured after each amplification cycle using a fluorescent readout. A typical real-time PCR amplification plot shows a sigmoidal-shaped curve (on a linear scale) and includes a baseline phase, followed by an exponential phase that reaches a plateau via a linear phase. The most efficient phase of amplification is represented by the exponential phase and the amount of amplified PCR products doubles with each cycle (at an amplification efficiency of 100%). A relative quantification of a target to a calibrator is enabled by real-time PCR. The “absolute” amount of target sequence in a qPCR reaction is measured relative to a standard curve, which is generated from a sample of known quantity or copy number. This method implies that the amplification efficiencies of the standards and the sample are equivalent. Differences in PCR efficiencies can significantly affect the accuracy of the quantification (Quan et al., 2018, MDPI, “dPCR: A Technology Review”).

qPCR is a widely used method for DNA quantification since it offers various advantages. Such advantages are, within others, the species-specific identification, a wide dynamic range as well as the ease of automation.

For the DNA quantification by qPCR a standard curve is needed to calculate DNA amount of the sample. Usually the standard curve is prepared by a serial dilution by the user. The preparation of the standard curve is critical for a precise DNA quantification of samples. Depending on the user's skills and quality of the used equipment for standard curve preparation, the process of DNA dilution and preparation of the DNA standard curve itself can be very tedious, laborious and error prone which can lead to wrong quantification results.

Another approach to quantify DNA makes use of a digital polymerase chain reaction (dPCR; also abbreviated as e.g. digital PCR, DigitalPCR, ddPCR or dePCR), which is a refinement of other polymerase chain reaction methods such as qPCR.

Digital polymerase chain reaction (dPCR) enables the absolute quantification of target nucleic acids present in a sample and alleviates the shortcomings of qPCR. Unlike qPCR, dPCR does not rely on calibration curves for sample quantification. Hence, it avoids the pitfalls associated with variations in reaction efficiencies. dPCR is a method of absolute nucleic acid quantification that hinges on the detection of end-point fluorescent signals and the enumeration of binomial events, i.e. absence or presence of fluorescence in a partition. In dPCR, the sample is first partitioned into many independent PCR sub-reactions such that each partition contains either a few, one or no target sequences. Such partitions or microreactors can be arranged for example, but not limited to, by small water-in-oil droplets or by microfluidic nanoplates.

After PCR, the concentration of the target sequence is quantified with a statistically defined accuracy using Poisson's statistics, wherein partitions with either 0, 1, or more target sequences are each needed for the calculations. Interestingly, sample partitioning efficiently concentrates the target sequences within the isolated microreactors. This concentration effect reduces template competition and thus enables the detection of rare mutations in a background of wild-type sequences (Quan et al., 2018, MDPI, “dPCR: A Technology Review”).

dPCR may also allow for a higher tolerance to inhibitors present in a sample, because there is no need to have an amplification efficiency per cycle of almost 100%, as required for qPCR. Instead, it is sufficient if at the end of the amplification reaction either a signal or no signal is detectable.

PCR carries out one reaction per single sample. dPCR also carries out a single reaction within a sample, however the sample is separated into a large number of partitions and the reaction is carried out in each partition individually. This separation allows a more reliable collection and sensitive measurement of nucleic acid amounts.

Instead of performing one reaction per well, dPCR involves partitioning the PCR solution into at least a few hundred, but in most cases several thousand or tens of thousands or more of nano-liter sized partitions, where a separate PCR reaction takes place in each one. A dPCR solution is made similarly to a quantitative assay either using fluorescence-quencher probes or intercalating dyes, and a PCR master mix, which contains DNA polymerase, dNTPs, MgCl2, and reaction buffers at optimal concentrations.

Several different methods can be used for sample partitioning, including microwell plates, microfluidic nanoplates, capillaries, oil emulsion, and arrays of miniaturized chambers with nucleic acid binding surfaces.

After multiple PCR amplification cycles, the samples are checked for fluorescence with a binary readout of “0” (absence) or “1” (presence). The fraction of fluorescing partitions is recorded. The partitioning of the sample allows one to estimate the number of different molecules by assuming that the molecule population follows the Poisson distribution, thus accounting for the possibility of multiple target molecules inhabiting a single partition.

In contrast to a qPCR reaction, a dPCR reaction is an endpoint PCR reaction. dPCR uses the number of fluorescence-positive partitions over the total to back-calculate the target concentration. In contrast to qPCR, calibration curves are not needed for sample quantification in dPCR. All in all, compared to qPCR, dPCR provides a more robust quantification, is less prone to inhibitors and is independent of a quantification standard.

The benefits of dPCR include increased precision through massive sample partitioning, which ensures reliable measurements in the desired DNA sequence due to reproducibility. Error rates are larger when detecting small-fold change differences with qPCR, while error rates are smaller with dPCR due to the smaller-fold change differences that can be detected in DNA sequence. Also, dPCR is highly quantitative as it does not rely on relative fluorescence of the solution to determine the amount of amplified target DNA.

Despite these advancements, current dPCR systems are limited by their partition capacity (often≤ 100.000) per sample due to physical limitations and manufacturing constraints, which in turn limits the dynamic range of dPCR systems (Shum et al., 2022, BioRxiv, “Next generation digital PCR: high dynamic range single molecule DNA counting via ultra-partitioning”), i.e. the dynamic range is determined by the total number of partitions (Jones et al., 2016, “Digital PCR dynamic range is approaching that of real-time quantitative PCR”). The dynamic range of detection as used herein is defined as the range of sample concentration within which quantitation can be performed in a digital PCR and, as mentioned above, is directly linked to the amount of available partitions per sample. Hence, the dynamic range is proportional to the number of partitions in a reaction (dMIQE Group and Huggett, 2020, Clin Chem., “The Digital MIQE Guidelines Update: Minimum Information for Publication of Quantitative Digital PCR Experiments for 2020”) and increasing the number of partitions extends the dynamic range (Basu, 2017, SLAS Technol., “Digital Assays Part I: Partitioning Statistics and Digital PCR”), so ideally, dPCR systems involve a significantly greater number of partitions to reduce or eliminate partition saturation, thereby increasing the quantitative precision and dynamic range (Shum et al., 2022, BioRxiv, “Next generation digital PCR: high dynamic range single molecule DNA counting via ultra-partitioning”). Consequently, in certain situations a prior knowledge of the template concentration may be required to avoid saturating the instrument. When concentrations are sufficiently high, commonly used methods, such as those that employ fluorometry and spectrophotometry, can be used to quantify nucleic acids and guide the dilution to concentrations for optimal measurement using dPCR. It should be noted that such methods estimate mass per unit volume of the component nucleic acid bases not the macromolecule. Consequently, determination of genome copies using approaches that measure mass requires knowledge, or assumptions, of template composition, purity, and quality to convert mass to moles. Users should also be aware of potential interfering factors that may disrupt the accuracy of such optical methods (dMIQE Group and Huggett, 2020, Clin Chem., “The Digital MIQE Guidelines Update: Minimum Information for Publication of Quantitative Digital PCR Experiments for 2020”).

The determination of the quantity of DNA recovered from forensic samples as well as from other samples is a critical step in the overall DNA typing process or in the detection of DNA in various other fields of science. In order to ensure that a positive result is a positive result and/or a negative result is a negative result due to the absence of DNA, quantification of DNA is of absolute importance. For example, a narrow range of input DNA from 0.5 to 2 ng is often needed to produce optimal results with for example multiplex DNA typing kits. The accurate measurement of the quantity of DNA in a sample is also important for the success of DNA profiling, e.g. via short tandem repeats (STRs). In fact, the more accurate the determination of the amount of amplifiable DNA, the more successful the DNA profiling.

A sensitive DNA quantification method based on digital PCR with a high dynamic range to accurately detect and quantify DNA for a wide range of disciplines and to overcome the limitation of digital PCR that the dynamic range is determined by the total number of partitions is therefore of great interest and there is a need for a method to encounter the drawbacks of the currently available kits and methods.

### SUMMARY OF THE INVENTION

The present invention solves the above identified problem and provides for the following solution as outlined below. In particular, the present invention provides for a method for extending the dynamic range (herein: the dynamic range of detection as used herein is defined as the range of sample concentration within which quantitation can be performed in a digital PCR without increasing the number of available partitions per sample per reaction, so that a larger range of samples can be accommodated without the need for dilution of the sample. Thereby, this method overcomes the above-mentioned drawbacks and limitations of digital PCR in terms of dynamic range. Surprisingly, and in contrast to the art as described above, the applicant developed a method, which can be used to extend the dynamic range for at least the factor of 10x.

In a first aspect, the present invention provides for a method of nucleic acid quantification, the method comprising the steps of
a. providing a sample comprising a plurality of nucleic acid molecules;
b. amplifying at least one single copy locus within said plurality of nucleic acid molecules, wherein the amplification method is a digital polymerase chain reaction;
c. amplifying at least one multicopy locus within said plurality of nucleic acid molecules, wherein the amplification method is a digital polymerase chain reaction;
d. detecting the amplification products of step b and c; and
e. quantifying the amount of the plurality of nucleic acid molecules present in the sample.

In a second aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one multicopy locus and at least one single copy locus and wherein the amplification method is a digital polymerase chain reaction method;
c. detecting the at least two amplification products; and
d. quantifying the amount of the plurality of nucleic acid molecules present in the sample.

The quantifiable nucleic acid concentration ranges for the at least one multicopy locus and the at least one single copy locus are overlapping. This increases or extends the dynamic range of the methods of the invention without the need of increasing the number of available partitions per sample per reaction.

Therefore, In a third aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one multicopy locus and at least one single copy locus and wherein the amplification method is a digital polymerase chain reaction method;
c. detecting the at least two amplification products; and
d. quantifying the amount of the plurality of nucleic acid molecules present in the sample;
   wherein the quantifiable nucleic acid concentration ranges for the at least one multicopy locus and the at least one single copy locus are overlapping.

In a fourth aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one multicopy locus and at least one single copy locus and wherein the amplification method is a digital polymerase chain reaction method;
c. detecting the at least two amplification products; and
d. quantifying the amount of the plurality of nucleic acid molecules present in the sample;
   wherein the quantifiable nucleic acid concentration ranges for the at least one multicopy locus and the at least one single copy locus are overlapping; and wherein the detection accuracy is at least 70%.

In a fifth aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one single copy locus and at least one multicopy locus, wherein the amplification method is the digital polymerase chain reaction method;
c. detecting the at least two amplification products; and
d. quantifying the amount of the plurality of nucleic acid molecules present in the sample;
   wherein the quantifiable nucleic acid concentration ranges for the at least one multicopy locus and the at least one single copy locus are overlapping;
   wherein if the concentration of the plurality of nucleic acid molecules present in the sample is low and the amplification of the at least one single copy locus does not result in a detectable amplification product, then the at least one multicopy locus amplification product is used for quantification;
   wherein if the concentration of the plurality of nucleic acid molecules present in the sample is high and signal saturation is reached for the at least one multicopy locus amplification product, then the at least one single copy locus amplification product is used for quantification.

In a sixth aspect, the present invention provides for a method of nucleic acid quantification, the method comprising the steps of
a. providing a sample comprising a plurality of nucleic acid molecules;
b. amplifying at least two genomic loci within said plurality of nucleic acid molecules, wherein the at least two genomic loci comprise at least one single copy locus and at least one multicopy locus, wherein the amplification method is the digital polymerase chain reaction method;
c. detecting the at least two amplification products; and
d. quantifying the amount of the plurality of nucleic acid molecules in the sample;
   wherein the quantifiable nucleic acid concentration ranges for the at least one multicopy locus and the at least one single copy locus are overlapping;
   wherein if the concentration of the plurality of nucleic acid molecules is lower than 0.002 ng/reaction, quantification is based solely on the steps performed for the at least one multicopy locus; and/or wherein if the concentration of the plurality of nucleic acid molecules is higher than 125 ng/reaction, quantification is based solely on the steps performed for the at least one single copy locus.

The sample can be distributed into a plurality of partitions prior to the amplification step and the method is performed in each of said plurality of partitions.

Optionally, the sample can be diluted prior to partitioning. This might be necessary in cases where, for example, signal saturation was reached in a first run of the method according to the invention due to a high nucleic acid concentration of said sample.

In several embodiments of each of the above aspects, one multicopy locus and one single copy locus are amplified within one dPCR partition. In several alternative embodiments of each of the above aspects, one multicopy locus and one single copy locus are amplified within different dPCR partitions. In several other embodiments of each of the above aspects, more than one multicopy locus and one single copy locus are amplified, either within one dPCR partition, or within different dPCR partitions. In several other embodiments of each of the above aspects, more than one single copy locus and one multicopy locus are amplified, either within one dPCR partition, or within different dPCR partitions. In several other embodiments of each of the above aspects, more than one multicopy locus and more than one single copy locus are amplified, either within one dPCR partition, or within different dPCR partitions.

In several embodiments of each of the above aspects, the at least one multicopy locus and the at least one single copy locus are amplified within one dPCR partition.

In several other embodiments of each of the above aspects, the at least one multicopy locus and the at least one single copy locus are amplified within different dPCR partitions.

In several embodiments of each of the above aspects, the maximum quantifiable nucleic acid concentration of the sample is defined by the maximum quantifiable nucleic acid concentration of the at least one single copy locus.

In several embodiments of each of the above aspects, the minimum quantifiable nucleic acid concentration of the sample is defined by the minimum quantifiable nucleic acid concentration of the multicopy locus of the at least one multicopy locus having most copies.

In several embodiments of each of the above aspects, the sample is selected from the group of samples comprising, but not limited to, eukaryotic-, human-, animal-, plant-, bacterial-, archaeal-, oomycetes-, viral-, mitochondrial-, genomic-, extrachromosomal-, gonosomal-, autosomal-, human-autosomal-, or fungal-DNA or RNA or cells, environmental samples (e.g. comprising microorganisms), or food samples (e.g. animal- or plant-derived), or mixtures thereof.

In several embodiments of each of the above aspects, the sample is a eukaryotic sample.

In several embodiments of each of the above aspects, the detection step is performed using at least two probes, wherein the probes bind to the at least two amplification products.

In several embodiments of each of the above aspects, the size of the amplification products is between 20 base pairs and 2000 base pairs long.

In several embodiments of each of the above aspects, the amplification is a triplex dPCR amplification of one single copy and two multicopy loci, wherein the two multicopy loci each occur in different copy numbers.

In several other embodiments of each of the above aspects, the amplification is a multiplex dPCR amplification of one single copy and a plurality of multicopy loci, wherein the plurality of multicopy loci each occur in different copy numbers.

According to several embodiments of each of the above aspects, the dPCR method is a nanoplate-based dPCR method or a water-in-oil droplet based dPCR method or a micro- or nanofluidic chip based dPCR method or any other dPCR method.

In several embodiments of each of the above aspects, the detection accuracy is at least 70%.

According to several embodiments of each of the above aspects, the method is used for forensic analyses.

In several embodiments of each of the above aspects, the at least one multicopy locus comprises a copy number per genome and/or transcriptome in the range of 2 to 260000, more preferable between 5 to 200000, even more preferable between 10 to 130000.

When the digital polymerase chain reaction comprises at least 8500 partitions, the amount of genomes and/or transcriptomes comprising at least one multicopy locus and at least one single copy locus is between 0,000024 to 42500.

When the digital polymerase chain reaction comprises at least 22000 partitions, the amount of genomes and/or transcriptomes comprising said at least one multicopy locus and said at least one single copy locus is between 0,00001 to 100000.

When the digital polymerase chain reaction comprises at least 26000 partitions, the amount of genomes and/or transcriptomes comprising at least one multicopy locus and at least one single copy locus is between 0,000008 to 130000.

In several embodiments of each of the above aspects, the lower end of the dynamic range of the digital amplification reaction is reached when one copy of a MCL with 130000 copies per genome and/or transcriptome is present in the digital amplification reaction.

The invention also relates to a kit for use in the methods according to the invention, the kit comprising
i. at least two primers and/or at least one probe for the amplification and/or quantification of at least one multicopy locus, and
ii. at least two primers and/or at least one probe for the amplification and/or quantification of at least one single copy locus.

### DETAILED DESCRIPTION OF THE INVENTION

In a first aspect, the present invention provides for a method of nucleic acid quantification, the method comprising the steps of
a. providing a sample comprising a plurality of nucleic acid molecules;
b. amplifying at least one single copy locus within said plurality of nucleic acid molecules, wherein the amplification method is a digital polymerase chain reaction;
c. amplifying at least one multicopy locus within said plurality of nucleic acid molecules, wherein the amplification method is a digital polymerase chain reaction;
d. detecting the amplification products of step b and c; and
e. quantifying the amount of the plurality of nucleic acid molecules present in the sample.

In a second aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one multicopy locus and at least one single copy locus and wherein the amplification method is a digital polymerase chain reaction method;
c. detecting the at least two amplification products; and
d. quantifying the amount of the plurality of nucleic acid molecules present in the sample.

The quantifiable nucleic acid concentration ranges for the at least one multicopy locus and the at least one single copy locus are overlapping. This increases or extends the dynamic range of the methods of the invention without the need of increasing the number of available partitions per sample per reaction.

In a third aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one multicopy locus and at least one single copy locus and wherein the amplification method is a digital polymerase chain reaction method;
c. detecting the at least two amplification products; and
d. quantifying the amount of the plurality of nucleic acid molecules present in the sample;
   wherein the quantifiable nucleic acid concentration ranges for the at least one multicopy locus and the at least one single copy locus are overlapping.

In a fourth aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one multicopy locus and at least one single copy locus and wherein the amplification method is a digital polymerase chain reaction method;
c. detecting the at least two amplification products; and
d. quantifying the amount of the plurality of nucleic acid molecules present in the sample;
   wherein the quantifiable nucleic acid concentration ranges for the at least one multicopy locus and the at least one single copy locus are overlapping; and wherein the detection accuracy is at least 70%.

In a fifth aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one multicopy locus and at least one single copy locus and wherein the amplification method is a digital polymerase chain reaction method;
c. detecting the at least two amplification products; and
d. quantifying the amount of the plurality of nucleic acid molecules present in the sample,
   wherein the quantifiable nucleic acid concentration ranges for the at least one multicopy locus and the at least one single copy locus are overlapping;
   wherein if the concentration of the plurality of nucleic acid molecules present in the sample is low and the amplification of the at least one single copy locus does not result in a detectable amplification product, then the at least one multicopy locus amplification product is used for quantification; wherein if the concentration of the plurality of nucleic acid molecules present in the sample is high and signal saturation is reached for the at least one multicopy locus amplification product, then the at least one single copy locus amplification product is used for quantification.

In a sixth aspect, the present invention provides for a method of nucleic acid quantification, the method comprising the steps of
a. providing a sample comprising a plurality of nucleic acid molecules;
b. amplifying at least two loci within said plurality of nucleic acid molecules, wherein the at least two loci comprise at least one single copy locus and at least one multicopy locus, wherein the amplification method is the digital polymerase chain reaction method;
c. detecting the at least two amplification products; and
d. quantifying the amount of the plurality of nucleic acid molecules present in the sample,
   wherein the quantifiable nucleic acid concentration ranges for the at least one multicopy locus and the at least one single copy locus are overlapping;
   wherein if the concentration of the plurality of nucleic acid molecules is lower than 0.002 ng/reaction, quantification is based solely on the steps performed for the at least one multicopy locus; and/or
   wherein if the concentration of the plurality of nucleic acid molecules is higher than 125 ng/reaction, quantification is based solely on the steps performed for the at least one single copy locus.

“Dynamic range” as used herein, is defined as the range of sample concentration within which quantitation can be performed in a digital PCR.

The term “plurality of nucleic acid molecules” includes desoxyribonucleic acids (DNA) and ribonucleic acids (RNA) or mixtures thereof. Naturally or artificially modified DNA and RNA is also encompassed.

The term “at least one” as used in the present invention refers to 1, 2, 3, or more. For example, at least one locus is meant to include 1, 2, 3, or more loci, respectively. Similarly, “at least two” as used herein refers to 2, 3, 4 or more. For example, at least loci is meant to include 2, 3, 4 or more loci, respectively.

The term “detectable amplification product” as used herein means that amplification was successful and the amplification product can be detected by means described herein (e.g. by probes) and the amount of the plurality of nucleic acid molecules present in the sample can be quantified. In contrast, in case the amplification product is not detectable, there may be several reasons for this scenario to occur. For example, the amplification may have failed due to too little amounts of nucleic acids per reaction. Along this line, detection might fail, if the signal intensity is not significantly higher than the background signal.

The term “quantifying the amount” as used herein refers to the determination or calculation of a concentration. It may, however, also comprise the determination of absolute amount values.

“Signal saturation” as used herein means that the concentration of the plurality of nucleic acid molecules is so high that most or all of the partitions show a positive signal. If this happens, the quantification cannot be performed precisely anymore.

The term “accuracy” as used herein refers to a measure of trueness. Accuracy is how close a given set of measurements are to their true value. It is a description of only systematic errors, a measure of statistical bias of a given measure of central tendency. Low accuracy causes a difference between a result and a true value.

“Accuracy” should not be confounded with “precision”, which defines how close the measurements are to each other. In other words, precision is a description of random errors, a measure of statistical variability.

In case the locus is a multicopy locus, it may be found many times in the genome or many times on a single chromosome. It may also be found on several of the analyzed DNA or RNA or cDNA molecules. In case the locus is a single copy locus, it may be present only once on a single chromosome (e.g. Y-chromosomal single copy locus) or twice in the genome (e.g. on both alleles of an autosome or an X-chromosomal single copy locus). It may also be found only once on the analysed DNA or RNA or cDNA molecules.

Exemplary multicopy loci comprise gonosomal and/or autosomal multicopy loci, non-limiting examples comprising MCL-Y, MCL-auto, repetitive DNA sequences such as Alu sequences, retrotransposons, or any other multicopy locus present on the plurality of nucleic acids of the sample analyzed.

Exemplary single copy loci comprise gonosomal and/or autosomal single copy loci, non-limiting examples comprising SRY, GRM7, ZFY, AMELY, TBL1Y, TSPY1, TSPY2, USP9Y, DDX3Y, UTY, TB4Y, EIF1AY, KDM5D, XKRY, HSFY1, HSFY2, PRY, PRY2, or any other single copy locus on the plurality of nucleic acids of the sample analyzed.

All the gene examples mentioned above are just exemplary genes and shall not be considered as limiting the scope of the invention. In fact, the methods of the invention can be performed using any multicopy locus (or even more than one) and any single copy locus (or even more than one), as long as the corresponding quantifiable nucleic acid concentration ranges are overlapping. The invention can also be performed by using at least one low copy multicopy locus instead of the at least one single copy locus and at least one MCL with a higher copy number than the low copy multicopy locus. In such cases, the low copy number locus has a maximum copy number between 2 and 20, preferable between 2 and 10 and even more preferable between 2 and 5.

Generally, the quantifiable range of nucleic acid molecules of a sample depends on the copy number of the loci analyzed. The higher the copy number of an analyzed locus, the lower the input material concentration that can be quantified. The lower the copy number of an analyzed locus, the higher the input material concentration that can be quantified.

Hence, knowledge about the copy number of a specific locus (either of both the SCL and the MCL or just the SCL) combined with the obtained number of positive and negative partitions is used to calculate the nucleic acid concentration of a sample.

However, the quantifiable nucleic acid concentration ranges for the at least one multicopy locus and the at least one single copy locus need to be overlapping. If, for example, one SCL and two MCLs are used, then the quantifiable nucleic acid concentration ranges of the SCL needs to overlap with the quantifiable nucleic acid concentration range of one of the MCLs (the one with less copy numbers) and said MCL needs to overlap with the second MCL having more copy numbers and so on for more MCLs.

In several embodiments of each of the aspects, the at last one multicopy locus comprises a copy number per genome and/or transcriptome in the range of 2 to 260000, more preferable between 5 to 200000, even more preferable between 10 to 130000.

When the digital polymerase chain reaction comprises at least 8500 partitions, the amount of genomes and/or transcriptomes comprising at least one multicopy locus and at least one single copy locus is between 0,000024 to 42500.

When the digital polymerase chain reaction comprises at least 22000 partitions, the amount of genomes and/or transcriptomes comprising said at least one multicopy locus and said at least one single copy locus is between 0,00001 to 100000.

When the digital polymerase chain reaction comprises at least 26000 partitions, the amount of genomes and/or transcriptomes comprising at least one multicopy locus and at least one single copy locus is between 0,000008 to 130000.

The maximum quantifiable nucleic acid concentration of the sample is defined by the maximum quantifiable nucleic acid concentration of the at least one single copy locus. The quantification may be limited by, for example, but not limited to, signal saturation.

The minimum quantifiable nucleic acid concentration of the sample is defined by the minimum quantifiable nucleic acid concentration of the multicopy locus of the at least one multicopy locus having most copies. The quantification may be limited by, for example, but not limited to, too few positive partitions.

In several embodiments of each of the above aspects, the lower end of the dynamic range of the digital amplification reaction is reached when one copy of a MCL with 130000 copies per genome and/or transcriptome is present in the digital amplification reaction.

The maximum distance between the highest and lowest quantifiable amplification target concentration according to this invention is the distance between the maximum quantifiable amount of the at least one SCL target until signal saturation is reached and the minimum quantifiable amount of the at least one MCL target. This is the case when at least 99,9% but less than 100% partitions are positive for the SCL target and at least one partition is positive for the at least one MCL target, wherein the minimum quantifiable amount of the at least one SCL target and the maximum quantifiable amount of the at least one MCL target are overlapping.

In several embodiments of each of the aspects, the sample is distributed into a plurality of partitions prior to the amplification step and the method is performed in each of said plurality of partitions. Optionally, the sample can be diluted prior to partitioning. This might be necessary in cases where, for example, signal saturation was reached in a first run of the method according to the invention due to a high nucleic acid concentration of said sample.

Herein, numerous different kinds of digital PCR may be used.

### Droplet Digital PCR

Droplet Digital PCR (ddPCR) is a method of dPCR in which a for example 20 microliter sample reaction including assay primers and either Taqman probes or an intercalating dye, is divided into about 20,000 nanoliter-sized oil droplets through a water-oil emulsion technique, thermocycled to endpoint in a 96-well PCR plate, and fluorescence amplitude read for all droplets in each sample well in a droplet flow cytometer.

### Chip-Based Digital PCR

Chip-based Digital PCR (dPCR) is also a method of dPCR in which the reaction mix (also when used in qPCR) is divided into at least a few hundred, but in most cases several thousand, or about 10,000 to about 45,000, or tens of thousands, or hundreds of thousand or more of partitions on a chip, then amplified using an endpoint PCR thermocycling machine, and is read using a high-powered camera reader with fluorescence filter for all partitions on each chip. Several fluorescent dyes may be used, as long as the corresponding fluorescent channels can be separated from each other. Examples for usable dyes comprise HEX, FAM, Cy5, Cy5.5 and Texas Red. Such a chip-based method relies on a nanofluidic chip.

QIAcuity plate based dPCR (QIAGEN) is preferred. This method is based on partitions in a plate for example and not oil drops. Here, ideally each partition that contains a target DNA molecule creates a signal, giving rise to no signal in partitions where no target DNA is, hence the word digital, yes/no.

According to several embodiments of each of the aspects, the dPCR method is a nanoplate-based dPCR method.

According to several embodiments of each of the aspects, the method is used for forensic analyses.

In several embodiments of each of the aspects, one multicopy locus and one single copy locus are amplified within one dPCR partition. In several alternative embodiments of each of the aspects, one multicopy locus and one single copy locus are amplified within different dPCR partitions. In several other embodiments of each of the aspects, more than one multicopy locus and one single copy locus are amplified, either within one dPCR partition, or within different dPCR partitions. In several other embodiments of each of the aspects, more than one single copy locus and one multicopy locus are amplified, either within one dPCR partition, or within different dPCR partitions. In several other embodiments of each of the aspects, more than one multicopy locus and more than one single copy locus are amplified, either within one dPCR partition, or within different dPCR partitions.

In several embodiments of each of the aspects, the at least one multicopy locus and the at least one single copy locus are amplified within one dPCR partition.

In several other embodiments of each of the aspects, the at least one multicopy locus and the at least one single copy locus are amplified within different dPCR partitions.

One or more multicopy loci occurring in 2 copies, or 10 copies, or 100 copies, or 1,000 copies or 10,000 copies, or 100,000 copies, or more than 100,000 copies, or between 2 and 11 copies, or between 101 and 1000 copies, or between 5,000 and 10,000 copies, or between 10,000 and 100,000 copies per cell can be used in the methods of the invention.

According to several embodiments of each of the aspects, the sample is selected from the group of samples comprising, but not limited to, eukaryotic-, human-, animal-, plant-, bacterial-, archaeal-, oomycetes-, viral-, mitochondrial-, genomic-, extrachromosomal-, gonosomal-, autosomal-, human-autosomal-, or fungal-DNA or RNA or cells, environmental samples (e.g. comprising microorganisms), or food samples (e.g. animal- or plant-derived), or mixtures thereof.

The methods according to the invention can also be used for human/animal pathogen testing (bacterial, fungal, oomycetes) and phytopathology (bacterial, fungal, oomycetes), using corresponding samples.

Preferably, the sample is a eukaryotic sample. Most preferably, the sample is a human sample. The sample may originate from one of the following sample or tissue types comprising whole blood, blood fractions, plasma, serum, body fluids, oral specimen, oral fluids, saliva, sputum, swab, urine, human biotic tissue, clothing samples containing biological material, vaginal swabs, sperm, skin or wound swabs or other samples containing biological material or other parts of the human body upon availability for isolation of nucleic acids. As used herein the terms “oral fluids” and “body fluids” refer to fluids that are excreted or secreted from the buccal cavity and from the body, respectively, from which nucleic acids can be isolated. As non-limiting examples, oral and body fluids may comprise saliva, sputum, swab, and urine.

In case of a forensic sample, the sample may be a male or a female sample. Alternatively, in case of a forensic sample, the sample may comprise a mixture of male and female nucleic acids, wherein the amount of female nucleic acids (e.g. DNA or RNA) exceeds the amount of male nucleic acids (e.g. DNA or RNA) by several orders of magnitude, e.g. in sexual assault samples or in blood samples of pregnant women comprising male fetal nucleic acids. Thus, according to another embodiment, the sample comprises one or more additional nucleic acids originating from a different individual.

According to several embodiments of each of the aspects, the size of each of the dPCR amplification products of the at least two loci may be between 20 base pairs and 2000 base pairs long.

The plurality of nucleic acid molecules in a sample and/or the probes (or other detection agent) and/or the amplification products can be labelled or can comprise modifications, such as base modifications, sugar modifications and/or backbone modifications and may also comprise fluorescent tags, barcodes, indices, peptides, proteins and/or chemical moieties attached to them.

In several embodiments of each of the aspects, the amplification is a triplex dPCR amplification of one single copy and two multicopy loci, wherein the two multicopy loci each occur in different copy numbers.

In several other embodiments of each of the aspects, the amplification is a multiplex dPCR amplification of one single copy and a plurality of multicopy loci, wherein the plurality of multicopy loci each occur in different copy numbers.

Multiplexing using a plurality of multicopy loci, each with a different copy number, increases the dynamic range even further than the use of one single copy locus and one multicopy locus.

The target loci, i.e. the single copy loci or multicopy loci may be located on autosomes or on gonosomes.

The dPCR amplification methods will comprise buffers, dNTPs or NTPs in addition to the enzymes required.

As used herein, the term “dNTP” refers to deoxyribonucleoside triphosphates. Non-limiting examples of such dNTPs are dATP, dGTP, dCTP, dTTP, dUTP, which may also be present in the form of labelled derivatives, for instance comprising a fluorescent label, a radioactive label, a biotin label. dNTPs with modified nucleotide bases are also encompassed, wherein the nucleotide bases are for example hypoxanthine, xanthine, 7-methylguanine, inosine, xanthinosine, 7-methylguanosine, 5,6-dihydrouracil, 5-methylcytosine, pseudouridine, dihydrouridine, 5-methylcytidine. Furthermore, ddNTPs of the above-described molecules are encompassed in the present invention.

As used herein, the term “NTP” refers to ribonucleoside triphosphates. Non-limiting examples of such NTPs are ATP, GTP, CTP, TTP, UTP, which may also be present in the form of labelled derivatives, for instance comprising a fluorescent label, a radioactive label, a biotin label.

According to another embodiment of of each of the aspects of the present invention, the amplification reaction comprises, (a) Tris-HCl at a pH of between 8 and 8.8 (at 20° C.) and/or, (b) potassium salt selected from the group of, potassium chloride and potassium sulphate and/or, (c) an ammonium salt, preferably ammonium chloride or ammonium sulphate and/or, (d) magnesium chloride and/or, (e) a hot-start polymerase.

Preferably, the concentration of Tris-HCl is in the range from 10 to 100 mM, most preferably in the range from 20 to 70 mM, the concentration of K⁺ is in the range from 1-25 mM, most preferred in the range from 2,5 to 20 mM, the concentration of NH₄⁺ in range from 1 to 40 mM, most preferred in the range from 2,5 to 30 mM, and a concentration of Mg²⁺ of 0.5 mM to 8 mM in excess to the concentration of the four dNTPs, most preferred a concentration of Mg²⁺of 0.7 mM to 5 mM in excess to the concentration of the four dNTPs, a hot-start polymerase, preferentially a hot-start polymerase allowing a hot-start time of less than 5 min, most preferred below 2 min.

The at least two amplification products can be detected by using DNA probes or RNA probes or DNA intercalating dyes or other known DNA labelling or detection agents. Detection of the amplification products may comprise direct or indirect fluorescent and/or chemical labelling and/or peptide—or protein-based labelling of the amplicon by a composition comprising one or more of the following: probes and/or guiding molecules and/or proteins and/or peptides and/or nucleic acids and/or derivatives of the aforementioned listed components. Preferably, the at least two amplification products are detected by using at least two probes, wherein at least one of the at least two probes binds to one of the at least two amplification products and at least one other probe of the at least two probes binds to another one of the at least two amplification products. Hence, the detection step is performed using at least two probes, wherein the probes bind to the at least two amplification products.

Non-limiting examples for probes comprise Taqman probes, Scorpion probes, Molecular-Beacon probes, and/or Fluorescence Resonance Energy Transfer Probes.

Quantification of the amount of the plurality of nucleic acid molecules in a sample is done by using Poisson's law of small numbers. The background section of this application provides a more detailed description of how the amounts can be quantified.

The invention also relates to a kit for use in the methods according to the invention, the kit comprising
i. at least two primers and/or at least one probe for the amplification and/or quantification of at least one multicopy locus, and
ii. at least two primers and/or at least one probe for the amplification and/or quantification of at least one single copy locus.

The kit according to the invention can further comprise a manual, buffers, and/or other reagents required for performing the methods of the invention.

The present methods of the invention provides for a short run time, good accuracy, high precision, low sensitivity to inhibitors and a high dynamic range.

The detection accuracy of the methods of the invention should be at least 95%, or at least 90%, or at least 85%, or at least 80%, or at least 75%, or, preferably, at least 70%. Alternatively, the detection accuracy of the methods of the invention may be between 70% and 100%, or between 70% and 99%, or between 71% and 99%, or between 70% and 98%, or between 70% and 97%, or between 70% and 96%, or between 70% and 95%, or between 70% and 90%, or between 70% and 85%, or between 70% and 80%, or between 75% and 99%, or between 80% and 99%, or between 85% and 99%, or between 90% and 99%.

Detection accuracy as used herein also comprises quantification accuracy and generally relates to the overall accuracy of the methods of the invention.

Calculation of Dynamic Range Extension in dPCR by Using a Combination of Single- and Multicopy Targets in Parallel

Current commercially available digital PCR systems are based on sample partitioning in microfluidic/nanofluidic cartridges or nanoplates or in water in oil droplets. For example for the QIAGEN QIAcuity dPCR systems there are nanoplates with 8500 or 26000 partitions per well available, the ThermoFisher QuantStudio 3D Digital PCR system is based on nanofluidic chips with 20000 partitions and the Bio-Rad QX200 Droplet Digital PCR system with 20000 droplets (partitions) per 20 μl sample.

The theoretical upper and lower end of the dynamic range can be calculated and expressed by Poisson statistics.

According to dMIQE Group and Huggett (dMIQE Group and Huggett, 2020, Clin Chem., “The Digital MIQE Guidelines Update: Minimum Information for Publication of Quantitative Digital PCR Experiments for 2020”), two assumptions for dPCR to fit the Poisson distribution are that all partitions are of equal volume, and that target molecules are randomly distributed across partitions. In practice, this means that each partition has an equal chance of containing target molecules. The number of target molecules present within positive partitions may be one, two, or more molecules, and it is currently impossible to determine how many molecules a given positive partition may contain. However, the number of molecules in a negative partition is known. If all partitions are of equal volume, the mean concentration of target molecules per partition (A) can be estimated from the probability that a partition is negative using the proportion of negative partitions and the Poisson distribution. This concentration is derived from the number of negative partitions (w) and the total number of partitions in the reaction (n):

$$λ = - ln ( \frac{w}{n} )$$

In the case when all partitions contain the amplification target, i.e. all partitions have a positive signal and the dPCR experiment is saturated, a quantification is not possible.

Thus, for the above mentioned dPCR systems the upper dynamic range is in theory reached when 8499 (=99,98%), 25999 (=99,99%) or 21999 (=99,99%) of the partitions contain at least one amplification target. Accordingly, for the above mentioned dPCR systems, λ would be (rounded to the nearest integer):

$${λ}_{8500} = - ln ( \frac{8500 - 8499}{8500} ) = 9$$

$${λ}_{26000} = - ln ( \frac{26000 - 25999}{26000} ) = 10$$

$${λ}_{22000} = - ln ( \frac{22000 - 21999}{22000} ) = 10$$

The above Δ values mark the statistical/theoretical limit of the invention. More practical, however, is a value of λ=5 in order to reduce the source of error.

For a DNA and/or RNA quantification system based on a single copy locus, the minimum amount of a genome and/or transcriptome per analyzed sample volume needed for one positive partition in a dPCR is one (=lower end of the dynamic range or lowest detectable DNA and/or RNA concentration in the dPCR experiment). The maximum amount of genomes and/or transcriptomes per analyzed sample volume before saturation is reached (m) is the number of copies per amplified locus per genome and/or transcriptome (c), wherein c is one in case of a single copy locus or one divided by the copy number of a multicopy locus in case a multicopy locus is amplified, multiplied by λ and multiplied by 99,98% (for 8500 partitions) or 99,99% (for 26000 or 22000 partitions) of the number of partitions (nₛₐₜᵤᵣₐₜᵢₒₙ):

$$m = c * λ * {n}_{saturation}$$

The lower end of the dynamic range in DNA and/or RNA quantification experiments by dPCR can be extended when in addition to an SCL at least one MCL is amplified in the dPCR. For example, in case that a MCL with one hundred copies per genome and/or transcriptome is amplified, it is in theory sufficient if the genome/transcriptome is present 0.01 times in order to arrive at one positive partition in the dPCR.

The terms “genome” and “transcriptome” refer to the entirety of DNA or RNA, respectively, per cell in the analyzed sample volume.

Further specifics of the good results obtained in the methods according to the invention are detailed in the Figure legends and Examples.

### EXAMPLES

The following examples are used in conjunction with the Figures and Tables to illustrate the invention.

Herein,
dPCR refers to digital PCR;
qPCR refers to real-time quantitative polymerase chain reaction.

All experiments were performed with QIAGEN digital PCR mixes in 20 μl reaction (total volume) on QIAcuity (nanoplate 8.5k 96-well) following the manufacturer's instructions. Any commercially available dPCR mix that allows for the digital amplification of SCL and MCL can be used in the methods of the invention, with non-limiting examples being the QIAcuity Residual DNA Quantification Kits, the QIAcuity UCP Probe PCR Kit, the QIAcuity EG PCR Kit, the QIAcuity Probe PCR Kit.

Amplification according to the invention took place for 40 cycles. The cycling protocol was as follows:

Initial Heat Activation Step at 95° C. for 3 minutes, followed by a 2-step amplification step at 95° C. for 5 seconds and 60° C. for 35 seconds.

The primers and probes shown in FIG. 5 were used for the experiments shown in all figures. However, the loci amplified and detected by these sequences and the sequences themselves shall not be regarded as limiting the scope of the invention, but shall just provide exemplary MCLs and SCLs that can be used in the methods of the invention. However, any MCL (at least one) as well as any SCL (at least one) can be used in the context of the present invention, as long as the corresponding quantifiable nucleic acid concentration ranges are overlapping.

The concentration ranges of the following paragraphs refer to human genomic DNA as the exemplarily used nucleic acid. While they illustrate well the dynamic range extension achieved by the methods of the invention, dynamic range extension can also be achieved using one or more single copy loci and one or more multicopy loci on other types of nucleic acids (e.g. RNA, cDNA, other DNA) and/or by using multiple and/or different MCLs and/or SCLs. The degree of dynamic range extension depends on several factors, for example, but not limited to, the type of nucleic acid used and/or the copy number or frequency of occurrence of the one or more multicopy loci used. Therefore, in order to provide an example of the methods of the invention, but without wishing to be bound by it, the following paragraphs refer to exemplary/specific embodiments for specific MCLs (MCL-auto or MCL-Y), specific SCLs (SCL-auto or SCL-Y) and human genomic DNA.

In specific embodiments of each of the aspects, the total measurable concentration of the plurality of nucleic acid molecules can range from 0.00001 ng/reaction to 2000 ng/reaction. Preferably, it ranges from 0.00005 ng/reaction to 1800 ng/reaction. More preferably, it ranges from 0.0001 ng/reaction to 1600 ng/reaction. Even more preferably, it ranges from 0.00019 ng/reaction to 1300 ng/reaction. Most preferably, it ranges from 0.00025 ng/reaction to 1000 ng/reaction.

In specific embodiments of each of the aspects, the concentration of the plurality of nucleic acid molecules measurable when amplifying a multicopy locus can range from 0.00001 ng/reaction to 100 ng/reaction. Preferably, it ranges from 0.00005 ng/reaction to 90 ng/reaction. More preferably, it ranges from 0.0001 ng/reaction to 80 ng/reaction. Even more preferably, it ranges from 0.00019 ng/reaction to 70 ng/reaction. Most preferably, it ranges from 0.00025 ng/reaction to 62.5 ng/reaction. The concentration of the plurality of nucleic acid molecules measurable when amplifying a multicopy locus can be less than 125 ng/reaction, or less than 66 ng/reaction, or less than 26 ng/reaction, or less than 2.6 ng/reaction, or less than 0.26 ng/reaction, or less than 0.026 ng/reaction, or less than 0.0026 ng/reaction, or less than 0.00026 ng/reaction.

In specific embodiments of each of the aspects, a detection accuracy of at least 70% can be reached when amplifying a multicopy locus at a concentration of the plurality of nucleic acid molecules ranging from 0.0039 ng/reaction to 40 ng/reaction. In specific embodiments of each of the aspects, a detection accuracy of at least 90% can be reached when amplifying a multicopy locus at a concentration of the plurality of nucleic acid molecules ranging from 0.125 ng/reaction to 40 ng/reaction (autosomal multicopy locus) or from 0.125 ng/reaction to 20 ng/reaction (gonosomal multicopy locus).

In specific embodiments of each of the aspects, the concentration of the plurality of nucleic acid molecules measurable when amplifying a single copy locus can range from 0.00001 ng/reaction to 2000 ng/reaction. Preferably, it ranges from 0.0001 ng/reaction to 1800 ng/reaction. More preferably, it ranges from 0.001 ng/reaction to 1600 ng/reaction. Even more preferably, it ranges from 0.0015 ng/reaction to 1300 ng/reaction. Most preferably, it ranges from 0.002 ng/reaction to 1000 ng/reaction. The concentration of the plurality of nucleic acid molecules measurable when amplifying a single copy locus can be more than 0.0005 ng/reaction, or more than 0.001 ng/reaction, or more than 0.0099 ng/reaction, or more than 0.099 ng/reaction, or more than 0.99 ng/reaction, or more than 9.9 ng/reaction, or more than 99 ng/reaction, or more than 999 ng/reaction.

In specific embodiments of each of the aspects, a detection accuracy of at least 70% can be reached when amplifying a single copy locus at a concentration of the plurality of nucleic acid molecules ranging from 0.125 ng/reaction to 1000 ng/reaction, or from 0.125 ng/reaction to 640 ng/reaction (autosomal single copy locus), or from 0.25 ng/reaction to 1000 ng/reaction (gonosomal single copy locus). In specific embodiments of each of the aspects, a detection accuracy of at least 90% can be reached when amplifying a single copy locus at a concentration of the plurality of nucleic acid molecules ranging from 1 ng/reaction to 160 ng/reaction (autosomal single copy locus), or from 2 ng/reaction to 80 ng/reaction (gonosomal single copy locus).

Table 1 shows the quantification data according to FIG. 1 and FIG. 2.

TABLE 1
Quantification data of FIG. 1 and FIG. 2

| | | Quantification in cp/μl | | | |
|---|---|---|---|---|---|
| | | MCL-auto | MCL-Y | SCL-auto | SCL-Y |
| NTC | | 0.00 | 0.00 | 0.00 | 0.00 |
| 0.00025 | ng/RxN | 0.09 | 0.09 | 0.00 | 0.00 |
| 0.0005 | ng/RxN | 0.20 | 0.19 | 0.00 | 0.00 |
| 0.002 | ng/RxN | 0.88 | 0.87 | 0.20 | 0.00 |
| 0.004 | ng/RxN | 1.44 | 1.94 | 0.00 | 0.00 |
| 0.008 | ng/RxN | 3.04 | 3.31 | 0.19 | 0.10 |
| 1.953125 | ng/RxN | 987.53 | 632.55 | 50.02 | 23.48 |
| 7.8125 | ng/RxN | 3988.70 | 2497.25 | 192.05 | 102.93 |
| 15.625 | ng/RxN | 7908.95 | 5102.40 | 373.70 | 198.65 |
| 31.25 | ng/RxN | 15035.70 | 9654.30 | 712.75 | 362.55 |
| 62.5 | ng/RxN | saturated | 18409.80 | 1399.60 | 706.95 |
| 125 | ng/RxN | saturated | saturated | 2673.00 | 1283.35 |
| 250 | ng/RxN | saturated | saturated | 5377.45 | 2734.95 |
| 500 | ng/RxN | saturated | saturated | 9652.00 | 4845.15 |
| 1000 | ng/RxN | saturated | saturated | 18702.65 | 8801.35 |

In specific embodiments of each of the aspects of the invention, for single copy loci the DNA concentration, which is still detectable and quantifiable, is at least 1000 ng/reaction without complete signal saturation.

In specific embodiments of each of the aspects of the invention, for multicopy loci the DNA concentration, which is still detectable and quantifiable is 0.00025 ng/reaction, potentially even less, without failure of quantification.

In specific embodiments of each of the aspects of the invention, there is also a range of concentration of the plurality of nucleic acid molecules for which both, the amplification of at least one single copy locus and at least one multicopy locus, can be performed and quantified. This range may comprise a concentration of the plurality of nucleic acid molecules from 0.002 ng/reaction to 62.5 ng/reaction, preferably from 0.002 ng/reaction to 31.25 ng/reaction, or from 0.008 ng/reaction to 31.25 ng/reaction or from 0.008 ng/reaction to 62.5 ng/reaction.

However, as mentioned above, other concentration ranges for other types of nucleic acids or for other SCLs or other (and optionally more than one) multicopy loci used with more or less copy numbers, respectively, are also encompassed within the methods of the invention. Generally, in accordance with the methods of the invention, the following applies: if the concentration of the plurality of nucleic acid molecules is too high, amplification may still be performed successfully, but quantification might not be successful due to signal saturation. If the concentration is too low, amplification, and/or detection, and/or quantification might fail. However, there is a certain input nucleic acid range which enables quantification by using both MCL and SCL, or either of them, thereby increasing the dynamic range of nucleic acid quantification.

### Example 1

Extension of Dynamic Range in dPCR by Using a Combination of Single- and Multicopy Autosomal Targets in Parallel

A dilution series of human DNA starting from 1000 ng/reaction to 0.00025 ng/reaction was generated. For copy number detection a single copy autosomal target SCL-auto and a multicopy autosomal target MCL-auto have been combined in a duplex dPCR reaction. DNA dilution samples have been run in 4 replicates for DNA concentrations from 0.00025 ng/R×N to 1.95 ng/R×N. For all higher DNA concentrations duplicates have been run. For 0.002 ng/reaction all 4 replicates have shown a signal. Quantification was possible until 31.25 ng/reaction (4 log) with multicopy target. All higher DNA concentrations could not be detected due to signal saturation. All partitions were positive. For the single copy target SCL-auto the highest DNA concentration detectable without complete signal saturation was 1000 ng/reaction without complete saturation. When applying both targets in parallel both targets have an overlapping range and the total range for the detection is from 0.002 ng/reaction to 1000 ng/reaction giving a 5 log dynamic range. The quantification results are illustrated in FIG. 1.

### Example 2

Extension of Dynamic Range in dPCR by Using a Combination of Single- and Multicopy Gonosomal Targets in Parallel

A dilution series of human male DNA starting from 1000 ng/reaction to 0.00025 ng/reaction was generated. For copy number detection a single copy Y-chromosomal target SCL-Y and a multicopy Y-chromosomal target MCL-Y have been combined in a duplex dPCR reaction. DNA dilution samples have been run in 4 replicates for DNA concentrations from 0.00025 ng/R×N to 1.95 ng/RxN. For all higher DNA concentrations duplicates have been run. For 0.002 ng/reaction all 4 replicates have shown a signal. Quantification was possible until 62.5 ng/reaction (4 log) with multicopy target. All higher DNA concentrations could not be detected due to signal saturation. All partitions were positive. For the single copy Y-chromosomal target SCL-Y the highest DNA concentration detectible was 1000 ng/reaction without complete signal saturation. When applying both targets in parallel both targets have an overlapping range and the total range for detection is from 0.002 ng/reaction to 1000 ng/reaction giving a 5 log dynamic range. The quantification results are illustrated in FIG. 2.

### Example 3

Dynamic Range Extension in dPCR by Using a Combination of Single- and Multicopy Autosomal Targets in Parallel and their Influence on Detection Accuracy

A dilution series of human DNA starting from 1000 ng/reaction to 0.002 ng/reaction was generated. For copy number detection a single copy autosomal target SCL-auto and a multicopy autosomal target MCL-auto have been combined in a duplex dPCR reaction. DNA dilution samples have been run in 4 replicates for the multicopy target and 4 replicates for the single copy target. The MCL-auto target shows a detection accuracy (detected vs. expected copy numbers) of more than 70% starting from 0.0039 ng/rxn to 40 ng/rxn with highest accuracy of more than 90% starting from 0.125 ng/rxn to 40 ng per reaction. The SCL-auto target shows an accuracy of more than 70% starting from 0.125 ng/rxn to 640 ng/rxn with highest accuracy of more than 90% starting from 1 ng/rxn to 160 ng/rxn. The MCL-auto detection system fails to report accurate copy numbers (accuracy of more than 70%) if more than 40 ng/rxn is used, while the second detection system is able to detect copy numbers for DNA amounts of up to 640 ng/rxn with an accuracy of more than 70%. Similar holds true for the lowest DNA amounts. Here the SCL-auto fails to report copy numbers with an accuracy of more than 70% below 0.125 ng/rxn while the MCL-auto is able to detect copy numbers down to 0.0039 ng/rxn with an accuracy of more than 70%. These data clearly demonstrate that the dynamic range can be extended when combining at least two detection systems with different optimal detection ranges. The quantification results are illustrated in FIG. 3.

### Example 4

Dynamic Range Extension in dPCR by Using a Combination of Single- and Multicopy Gonosomal Targets in Parallel and their Influence on Detection Accuracy

A dilution series of human DNA starting from 1000 ng/reaction to 0.002 ng/reaction was generated. For copy number detection a single copy gonosomal target SCL-Y and a multicopy gonosomal target MCL-Y have been combined in a duplex dPCR reaction. DNA dilution samples have been run in 4 replicates for the multicopy target and 4 replicates for the single copy target. The MCL-Y target shows a detection accuracy (detected vs. expected copy numbers) of more than 70% starting from 0.0039 ng/rxn to 40 ng/rxn with highest accuracy over 90% starting from 0.125 ng/rxn to 20 ng per reaction. The SCL-Y target shows an accuracy of more than 70% starting from 0.25 ng/rxn to 1000 ng/rxn with highest accuracy of more than 90% starting from 2 ng/rxn to 80 ng/rxn. The MCL-Y detection system fails to report accurate copy numbers (accuracy of more than 70%) if more than 40 ng/rxn is used, while the second detection system is able to detect copy numbers for DNA amounts of up to 1000 ng/rxn with an accuracy of more than 70%. Similar holds true for the lowest DNA amounts. Here the SCL-Y fails to report copy numbers with an accuracy of more than 70% below 0.25 ng/rxn while the MCL-Y is able to detect copy numbers down to 0.0039 ng/rxn with an accuracy of more than 70%. These data clearly demonstrate that the dynamic range can be extended when combining at least two detection systems with different optimal detection ranges. The quantification results are illustrated in FIG. 4.

### Example 5

Table 2 shows the calculation of the extension of the dynamic range for DNA and/or RNA quantification in a dPCR using a combination of the amplification of a single copy locus (SCL) and at least one multicopy locus (MCL) with two (MCL-2) or up to 300000 (MCL-300.000) copies per genome and/or transcriptome and per analyzed sample volume in dPCR systems with 8500 partitions (Tab.2 A), 26000 partitions (Tab.2 B) or 20000 partitions (Tab.2 C).

TABLE 2 A
For DNA and/or RNA quantification in dPCR based on a single copy locus (SCL) in a dPCR
system with 8500 partitions, the upper end of the dynamic range is reached when 76484 (λ =
9; black background colour) or 42491 (λ = 5; dark grey background colour) genomes and/or
transcriptomes comprising said single copy locus per analyzed sample are present in the
dPCR experiment and the lower end of the dynamic range is reached when one single genome
and/or transcriptome comprising said single copy locus per analyzed sample is present in the
dPCR experiment (grey background colour).
The combination of the amplification of said SCL with the amplification of at least
one multicopy locus (MCL) extends the lower end of the dynamic range dramatically in dPCR
DNA and/or RNA quantification assays given that both the dynamic range of the SCL and the
MCL are overlapping. In the present example, the lowest possible end of the dynamic
range is reached when one copy of a MCL with 76484 (λ = 9) or 42491 (λ = 5)
copies per genome and/or transcriptome is amplified (black background colour) in the dPCR.

| | | | | Practical maximum |
|---|---|---|---|---|
| | | | Theoretical maximum | (λ = 5) amount of |
| | | | (λ = 9) amount of | genomes and/or |
| | | Amount of genomes | genomes and/or | transcript-tomes |
| | | and/or transcriptomes | transcript-tomes per | per analyzed |
| | | per analyzed sample | analyzed sample | sample volume |
| | | volume needed for 1 | volume until 99.98% | until 99.98% of |
| | Copies per | positive partition | of saturation (upper | saturation (upper |
| | genome and/or | (lower end of dynamic | end of dynamic | end of dynamic |
| Target | transcriptome | range) | range) | range) |
| SCL | 1 | 1 | 76484.7 | 42491.5 |
| MCL-2 | 2 | 0.5 | 38242.4 | 21245.8 |
| MCL-10 | 10 | 0.1 | 7648.5 | 4249.2 |
| MCL-20 | 20 | 0.05 | 3824.2 | 2124.6 |
| MCL-100 | 100 | 0.01 | 764.8 | 424.9 |
| MCL-1000 | 1000 | 0.001 | 76.5 | 42.5 |
| MCL-10.000 | 10000 | 0.0001 | 7.6 | 4.2 |
| MCL-42491 | 42491 | 0.000024 | 1.8 | 1.0 |
| MCL-76484 | 76484 | 0.00001307 | 1.0 | 0.6 |
| MCL-100.000 | 100000 | 0.00001 | 0.8 | 0.4 |

TABLE 2 B
For DNA and/or RNA quantification in dPCR based on a single copy locus (SCL)
in a dPCR system with 26000 partitions, the upper end of the dynamic range is
reached when 259974 (λ = 10; black background colour) or 129987 (λ =
5; dark grey background colour) genomes and/or transcriptomes comprising said
single copy locus per analyzed sample are present in the dPCR experiment and
the lower end of the dynamic range is reached when one single genome and/or
transcriptome comprising said single copy locus per analyzed sample is present
in the dPCR experiment (grey background colour).
The combination of the amplification of said SCL with the amplification of
at least one multicopy locus (MCL) extends the lower end of the dynamic
range dramatically in dPCR DNA and/or RNA quantification assays given that
both the dynamic range of the SCL and the MCL are overlapping. In the present
case, the lowest possible end of the dynamic range is reached when one copy
of a MCL with 259974 (λ = 10) or 129987 (λ = 5) copies per genome
and/or transcriptome is amplified (black background colour) in the dPCR.

| | | | | Practical maximum |
|---|---|---|---|---|
| | | | Theoretical maximum | (λ = 5) amount of |
| | | | (λ = 10) amount of | genomes and/or |
| | | Amount of genomes | genomes and/or | transcript-tomes |
| | | and/or transcriptomes | transcript-tomes per | per analyzed |
| | | per analyzed sample | analyzed sample | sample volume |
| | | volume needed for 1 | volume until 99.98% | until 99.99% of |
| | Copies per | positive partition | of saturation (upper | saturation (upper |
| | genome and/or | (lower end of dynamic | end of dynamic | end of dynamic |
| Target | transcriptome | range) | range) | range) |
| SCL | 1 | 1 | 259974.0 | 129987.0 |
| MCL-2 | 2 | 0.5 | 129987.0 | 64993.5 |
| MCL-10 | 10 | 0.1 | 25997.4 | 12998.7 |
| MCL-20 | 20 | 0.05 | 12998.7 | 6499.4 |
| MCL-100 | 100 | 0.01 | 2599.7 | 1299.9 |
| MCL-1000 | 1000 | 0.001 | 260.0 | 130.0 |
| MCL-10.000 | 10000 | 0.0001 | 26.0 | 13.0 |
| MCL-100.000 | 100000 | 0.00001 | 2.6 | 1.3 |
| MCL-129987 | 129987 | 0.000008 | 2.0 | 1.0 |
| MCL-259974 | 259974 | 0.000004 | 1.0 | 0.5 |
| MCL-300.000 | 300000 | 0.000003 | 0.9 | 0.4 |

TABLE 2 C
For DNA and/or RNA quantification in dPCR based on a single copy locus (SCL)
in a dPCR system with 22000 partitions, the upper end of the dynamic range is
reached when 179964 (λ = 10; black background colour) or 99980 (λ = 5;
dark grey background colour) genomes and/or transcriptomes comprising said single copy
locus per analyzed sample are present in the dPCR experiment and the lower end of
the dynamic range is reached when one single genome and/or transcriptome comprising said
single copy locus per analyzed sample is present in the dPCR experiment (grey background colour).
The combination of the amplification of said SCL with the amplification of at least one
multicopy locus (MCL) extends the lower end of the dynamic range dramatically in dPCR DNA
and/or RNA quantification assays given that both the dynamic range of the SCL and the MCL
are overlapping. In the present case, the lowest possible end of the dynamic range
is reached when one copy of a MCL with 179964 (λ = 10) or 99980 (λ = 5) copies
per genome and/or transcriptome is amplified (black background colour) in the dPCR.

| | | | | Practical maximum |
|---|---|---|---|---|
| | | | Theoretical | (λ = 5) amount of |
| | | | maximum (λ = 10) | genomes and/or |
| | | Amount of genomes | amount of genomes | transcript-tomes |
| | | and/or transcriptomes | and/or transcript- | per analyzed |
| | | per analyzed sample | tomes per analyzed | sample volume |
| | Copies per | volume needed for 1 | sample volume until | until 99.99% of |
| | genome | positive partition | 99.98% of saturation | saturation (upper |
| | and/or | (lower end of dynamic | (upper end of | end of dynamic |
| Target | transcriptome | range) | dynamic range) | range) |
| SCL | 1 | 1 | 179964.0 | 99980.0 |
| MCL-2 | 2 | 0.5 | 89982.0 | 49990.0 |
| MCL-10 | 10 | 0.1 | 17996.4 | 9998.0 |
| MCL-20 | 20 | 0.05 | 8998.2 | 4999.0 |
| MCL-100 | 100 | 0.01 | 1799.6 | 999.8 |
| MCL-1000 | 1000 | 0.001 | 180.0 | 100.0 |
| MCL-10.000 | 10000 | 0.0001 | 18.0 | 10.0 |
| MCL-99.800 | 99800 | 0.00001 | 1.8 | 1.0 |
| MCL-179.964 | 179964 | 0.000006 | 1.0 | 0.6 |
| MCL-200.000 | 200000 | 0.000005 | 0.9 | 0.5 |

### Example 6

Extension of Dynamic Range in dPCR by Using a Combination of a Single Copy and at Least Two Multicopy Autosomal Targets in Parallel.

A dilution series of human DNA starting from 500 ng/reaction to 0.00000008192 ng/reaction was generated. For copy number detection a single copy autosomal target SCL-auto and two multicopy autosomal targets (medium-MCL-auto and high-MCL-auto) have been combined in a triplex dPCR reaction. DNA dilution samples have been run in 3 replicates for all DNA concentrations. Reliable quantification was possible from 0,00001024 ng/reaction until 0,8 ng/reaction (4 log) with high-multicopy-target (high-MCL-auto). All higher DNA concentrations could not be detected due to signal saturation. All partitions were positive. For the medium-multicopy-target (medium-MCL-auto) reliable quantification was possible from 0,00128 ng/reaction up to 20 ng/reaction (4 log) until saturation is reached. With the single-copy-target (SCL-auto) reliable quantification was possible within a range of 0,032 ng/reaction up to 500 ng/reaction (4 log). When applying all three targets in parallel all targets have overlapping ranges and the total range for the detection is from 0,00001024 ng/reaction to 500 ng/reaction giving a 7 log dynamic range. The quantification results are illustrated in FIG. 7.

### FIGURE LEGENDS

FIG. 1: Extension of dynamic range in digital PCR using a combination of targets with different copy numbers.

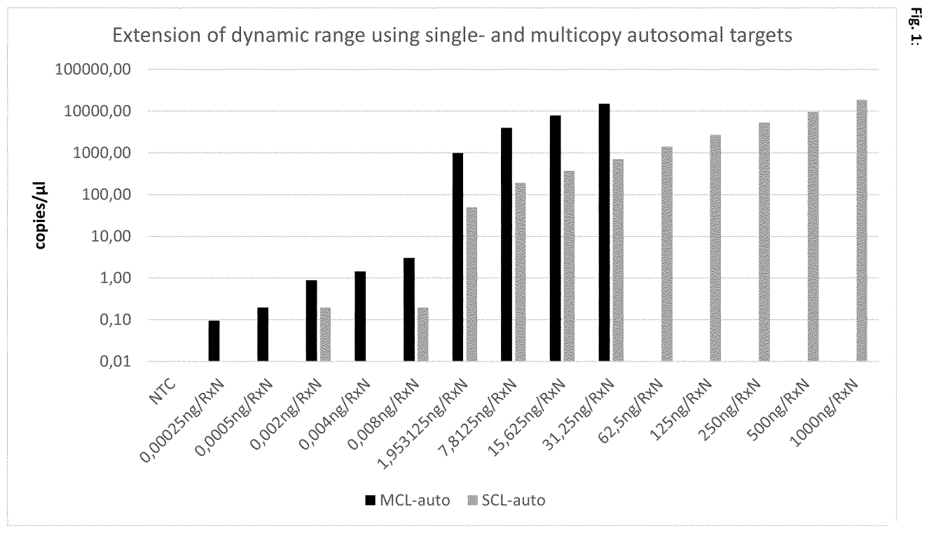

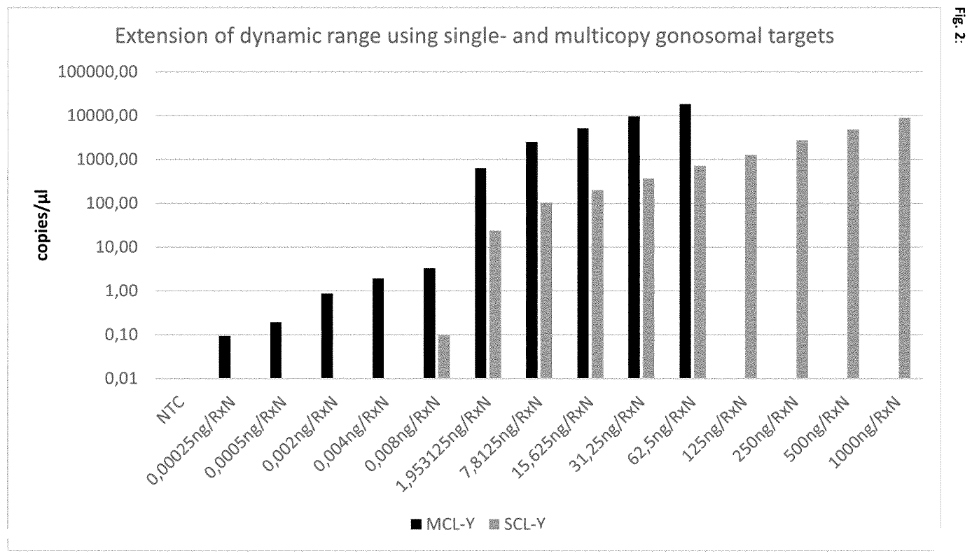

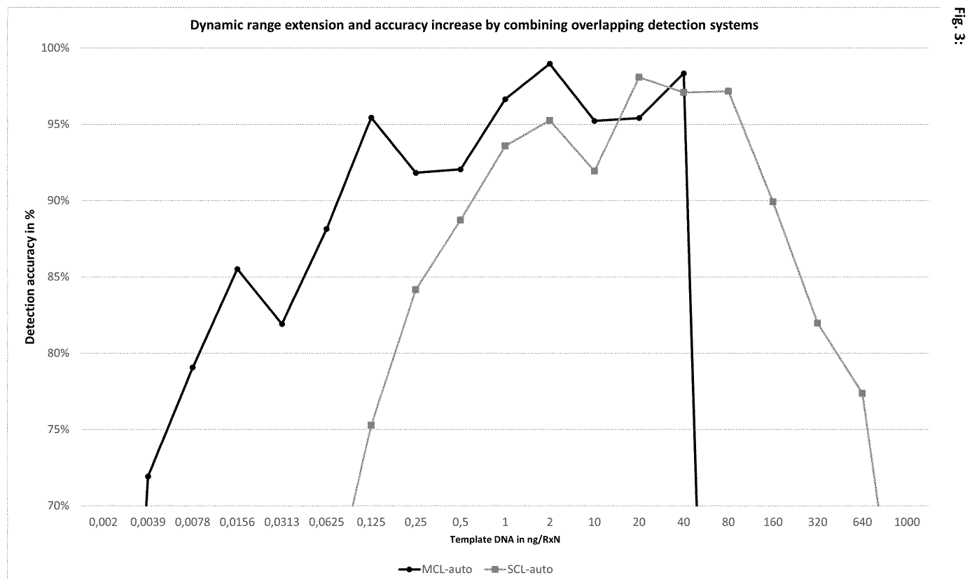

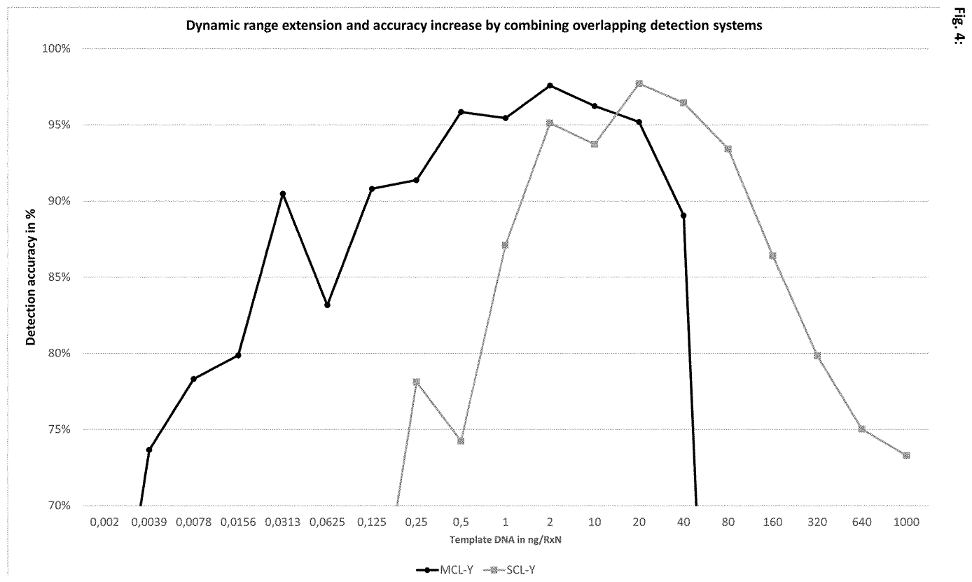

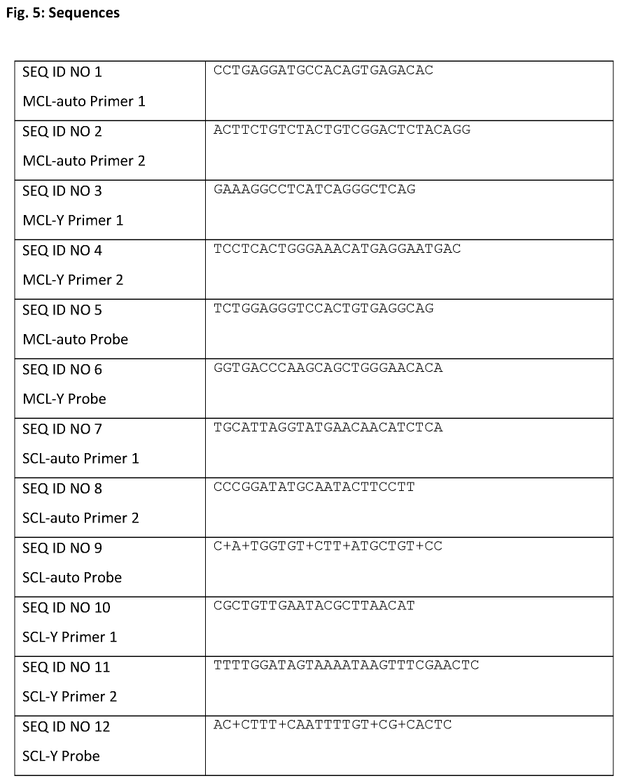

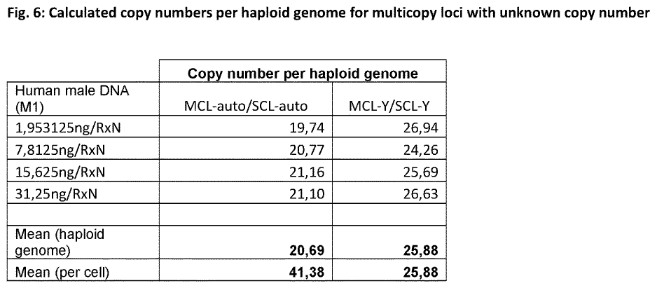

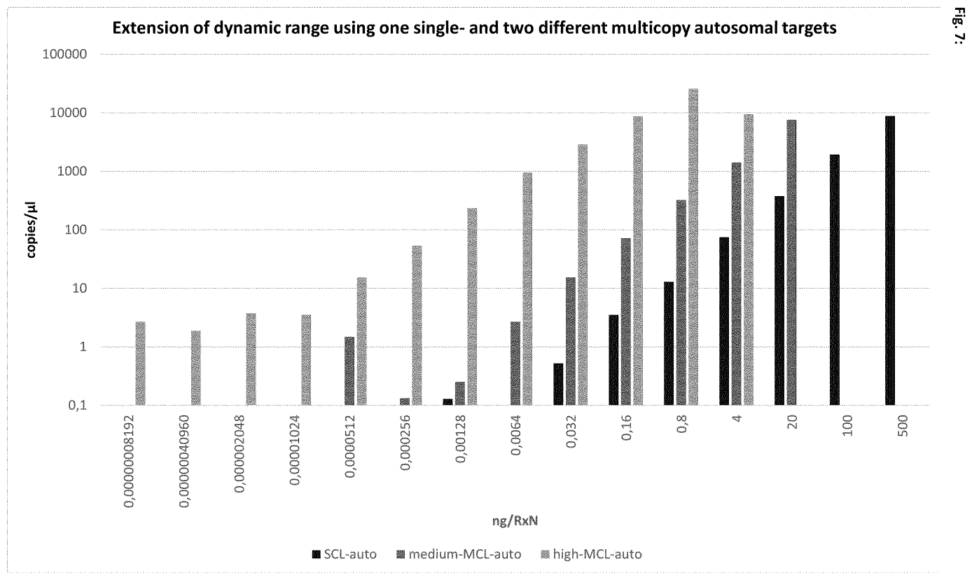

Shown are the DNA quantification data for a dilution series of human DNA starting from 0.00025 ng/reaction to 1000 ng/reaction. For copy number detection a single copy autosomal target SCL-auto and a multicopy autosomal target MCL-auto have been combined in a duplex reaction. DNA dilution samples have been run in 4 replicates for DNA concentrations from 0.00025 ng/R×N to 1.95 ng/RxN. For all higher DNA concentrations duplicates have been run. For 0.002 ng/reaction all 4 replicates have shown a signal. Quantification was possible until 31.25 ng/reaction (4 log) with multicopy target. All higher DNA concentrations could not be detected due to signal saturation. All partitions were positive. For the single copy target SCL-auto the highest DNA concentration detectable without complete signal saturation was 1000 ng/reaction without complete saturation. When applying both targets in parallel both targets have an overlapping range and the total range for the detection is from 0.002 ng/reaction to 1000 ng/reaction giving a 5 log dynamic range.

FIG. 2: Extension of dynamic range in digital PCR using a combination of targets with different copy numbers.

Shown are the DNA quantification data for a dilution series of human male DNA starting from 0.00025 ng/reaction to 1000 ng/reaction. For copy number detection a single copy Y-chromosomal target SCL-Y and a multicopy Y-chromosomal target MCL-Y have been combined in a duplex reaction. DNA dilution samples have been run in 4 replicates for DNA concentrations from 0.00025 ng/R×N to 1.95 ng/RxN. For all higher DNA concentrations duplicates have been run. For 0.002 ng/reaction all 4 replicates have shown a signal. Quantification was possible until 62.5 ng/reaction (4 log) with multicopy target. All higher DNA concentrations could not be detected due to signal saturation. All partitions were positive. For the single copy Y-chromosomal target SCL-Y the highest DNA concentration detectible was 1000 ng/reaction without complete signal saturation. When applying both targets in parallel both targets have an overlapping range and the total range for detection is from 0.002 ng/reaction to 1000 ng/reaction giving a 5 log dynamic range.

FIG. 3: Dynamic range extension in digital PCR by using a combination of single- and multicopy targets in parallel and their influence on detection accuracy.

Shown are the DNA quantification data for a dilution series of human DNA starting from 0.002 ng/reaction to 1000 ng/reaction. For copy number detection a single copy autosomal target SCL-auto and a multicopy autosomal target MCL-auto have been combined in a duplex reaction. DNA dilution samples have been run in 4 replicates for the multicopy target and 4 replicates for the single copy target. Depicted is the detection accuracy above 70% (detected vs. expected copy numbers) for the DNA template amounts tested. The MCL-auto target shows an accuracy of more than 70% starting from 0.0039 ng/rxn to 40 ng/rxn with highest accuracy over 90% starting from 0.125 ng/rxn to 40 ng per reaction. The SCL-auto target shows an accuracy of more than 70% starting from 0.125 ng/rxn to 640 ng/rxn with highest accuracy of more than 90% starting from 1 ng/rxn to 160 ng/rxn. The MCL-auto detection system fails to report accurate copy numbers (accuracy of more than 70%) if more than 40 ng/rxn is used, while the second detection system is able to detect copy numbers for DNA amounts of up to 640 ng/rxn with an accuracy of more than 70%. Similar holds true for the lowest DNA amounts. Here the SCL-auto fails to report copy numbers with an accuracy of more than 70% below 0.125 ng/rxn while the MCL-auto is able to detect copy numbers down to 0.0039 ng/rxn with an accuracy of more than 70%. These data clearly demonstrate that the dynamic range can be extended when combining at least two detection systems with different optimal detection ranges.

FIG. 4: Dynamic range extension in digital PCR by using a combination of single- and multicopy-targets in parallel and their influence on detection accuracy.

Shown are the DNA quantification data for a dilution series of human DNA starting from 0.002 ng/reaction to 1000 ng/reaction. For copy number detection a single copy gonosomal target SCL-Y and a multicopy gonosomal target MCL-Y have been combined in a duplex reaction. DNA dilution samples have been run in 4 replicates for the multicopy target and 4 replicates for the single copy target. Depicted is the detection accuracy above 70% (detected vs. expected copy numbers) for the DNA template amounts tested. The MCL-Y target shows an accuracy of more than 70% starting from 0.0039 ng/rxn to 40 ng/rxn with highest accuracy over 90% starting from 0.125 ng/rxn to 20 ng per reaction. The SCL-Y target shows an accuracy of more than 70% starting from 0.25 ng/rxn to 1000 ng/rxn with highest accuracy of more than 90% starting from 2 ng/rxn to 80 ng/rxn. The MCL-Y detection system fails to report accurate copy numbers (accuracy of more than 70%) if more than 40 ng/rxn is used, while the second detection system is able to detect copy numbers for DNA amounts of up to 1000 ng/rxn with an accuracy of more than 70%. Similar holds true for the lowest DNA amounts. Here the SCL-Y fails to report copy numbers with an accuracy of more than 70% below 0.25 ng/rxn while the MCL-Y is able to detect copy numbers down to 0.0039 ng/rxn with an accuracy of more than 70%. These data clearly demonstrate that the dynamic range can be extended when combining at least two detection systems with different optimal detection ranges.

FIG. 5: Sequences

Shown are exemplary sequences for primers and probes for use in the methods according to the invention. The “+” sign indicates that the base following the sign is a locked nucleic acid nucleotide (LNA). LNAs increase template binding strength.

FIG. 6: Calculated copy numbers per haploid genome for multicopy loci with unknown copy number Shown is the calculated copy number of a target multicopy locus (MCL-auto or MCL-Y) over the respective single copy locus copy number. Using Poisson's law of small numbers, the number of positive and negative partitions was used to calculate the concentration of the amplified loci and to back-calculate therefrom the copy number of the MCLs used, based on the knowledge that the SCL occurs once per haploid genome. These results demonstrate that the methods of the invention provide for a constant and accurate quantification over a large range (at least from about 1.9 ng/reaction to about 31.3 ng/reaction) of input sample nucleic acid concentration.

FIG. 7: Extension of dynamic range in dPCR by using a combination of a single copy and at least two multicopy autosomal targets in parallel. A dilution series of human DNA starting from 500 ng/reaction to 0.00000008192 ng/reaction was generated. For copy number detection a single copy autosomal target SCL-auto and two multicopy autosomal targets (medium-MCL-auto and high-MCL-auto) have been combined in a triplex dPCR reaction. DNA dilution samples have been run in 3 replicates for all DNA concentrations. Reliable quantification was possible from 0,00001024 ng/reaction until 0,8 ng/reaction (4 log) with high-multicopy-target (high-MCL-auto). All higher DNA concentrations could not be detected due to signal saturation. All partitions were positive. For the medium-multicopy-target (medium-MCL-auto) reliable quantification was possible from 0,00128 ng/reaction up to 20 ng/reaction (4 log) until saturation is reached. With the single-copy-target (SCL-auto) reliable quantification was possible within a range of 0,032 ng/reaction up to 500 ng/reaction (4 log). When applying all three targets in parallel all targets have overlapping ranges and the total range for the detection is from 0,00001024 ng/reaction to 500 ng/reaction giving a 7 log dynamic range. The quantification results are illustrated in this figure.

16. A method of nucleic acid quantification, the method comprising the steps of:
providing a sample comprising a plurality of nucleic acid molecules;
amplifying at least two loci within said plurality of nucleic acid molecules to produce at least two amplification products, wherein the at least two loci comprise at least one multicopy locus and at least one single copy locus, and wherein the amplification method is a digital polymerase chain reaction method;
detecting the at least two amplification products; and
quantifying the amount of the plurality of nucleic acid molecules present in the sample;
wherein the quantifiable nucleic acid concentration ranges for the at least one multicopy locus and the at least one single copy locus are overlapping.

17. The method according to claim 16, wherein the sample is distributed into a plurality of partitions prior to the amplification step, and wherein the method is performed in each of said plurality of partitions.

18. The method according to claim 16, wherein the maximum quantifiable nucleic acid concentration of the sample is defined by the maximum quantifiable nucleic acid concentration of the at least one single copy locus.

19. The method according to claim 16, wherein the minimum quantifiable nucleic acid concentration of the sample is defined by the minimum quantifiable nucleic acid concentration of the multicopy locus of the at least one multicopy locus having most copies.

20. The method according to claim 16, wherein the sample is a eukaryotic sample, a human sample, an animal sample, a plant sample, a bacterial sample, an archaeal sample, an oomycetes sample, a viral sample, a mitochondrial sample, a genomic sample, an extrachromosomal sample, a gonosomal sample, an autosomal sample, a human-autosomal sample, a fungal sample, DNA, RNA, cells, an environmental sample, a food sample or mixtures of two or more thereof.

21. The method according to claim 16, wherein the detection step is performed using at least two probes, and wherein the at least two probes bind to the at least two amplification products, respectively.

22. The method according to claim 16, wherein the size of the at least two amplification products is between 20 base pairs and 2,000 base pairs long.

23. The method according to claim 16, wherein the amplification is a triplex dPCR amplification of one single copy and two multicopy loci, and wherein the two multicopy loci each occur in different copy numbers.

24. The method according to claim 16, wherein the dPCR method is a nanoplate-based dPCR method.

25. The method according to claim 16, wherein the method is used for forensic analyses.

26. The method according to claim 16, wherein the at last one multicopy locus comprises a copy number per genome and/or transcriptome in the range of 2 to 260,000.

27. The method according to claim 16, wherein the at last one multicopy locus comprises a copy number per genome and/or transcriptome in the range of 5 to 200,000.

28. The method according to claim 16, wherein the at last one multicopy locus comprises a copy number per genome and/or transcriptome in the range of 10 to 130,000.

29. The method according to claim 17, wherein when the digital polymerase chain reaction comprises at least 8,500 partitions, and wherein the amount of genomes and/or transcriptomes comprising at least one multicopy locus and at least one single copy locus is between 0.000024 to 42,500.

30. The method according to claim 16, wherein when the digital polymerase chain reaction comprises at least 20,000 partitions, and wherein the amount of genomes and/or transcriptomes comprising said at least one multicopy locus and said at least one single copy locus is between 0.00001 to 100,000.

31. The method according to claim 16, wherein when the digital polymerase chain reaction comprises at least 26,000 partitions, and wherein the amount of genomes and/or transcriptomes comprising at least one multicopy locus and at least one single copy locus is between 0.000008 to 130,000.

32. A kit for use in the method according to claim 16, the kit comprising:
(i) at least two primers and/or at least one probe for the amplification and/or the quantification of at the least one multicopy locus; and
(ii) at least two primers and/or at least one probe for the amplification and/or the quantification of at the least one single copy locus.


SEQUENCE LISTING (12 sequences)

SEQ ID NO: 1 - DNA, 24 bp, synthetic construct
  source 1..24: mol_type = other DNA
cctgaggatgccacagtgagacac

SEQ ID NO: 2 - DNA, 28 bp, synthetic construct
  source 1..28: mol_type = other DNA
acttctgtctactgtcggactctacagg

SEQ ID NO: 3 - DNA, 22 bp, synthetic construct
  source 1..22: mol_type = other DNA
gaaaggcctcatcagggctcag

SEQ ID NO: 4 - DNA, 27 bp, synthetic construct
  source 1..27: mol_type = other DNA
tcctcactgggaaacatgaggaatgac

SEQ ID NO: 5 - DNA, 24 bp, synthetic construct
  source 1..24: mol_type = other DNA
tctggagggtccactgtgaggcag

SEQ ID NO: 6 - DNA, 25 bp, synthetic construct
  source 1..25: mol_type = other DNA
ggtgacccaagcagctgggaacaca

SEQ ID NO: 7 - DNA, 25 bp, synthetic construct
  source 1..25: mol_type = other DNA
tgcattaggtatgaacaacatctca

SEQ ID NO: 8 - DNA, 22 bp, synthetic construct
  source 1..22: mol_type = other DNA
cccggatatgcaatacttcctt

SEQ ID NO: 9 - DNA, 20 bp, synthetic construct
  source 1..20: mol_type = other DNA
  modified_base 2: mod_base = OTHER; note = locked nucleic acid (LNA) nucleotide
  modified_base 3: mod_base = OTHER; note = locked nucleic acid (LNA) nucleotide
  modified_base 9: mod_base = OTHER; note = locked nucleic acid (LNA) nucleotide
  modified_base 12: mod_base = OTHER; note = locked nucleic acid (LNA) nucleotide
  modified_base 19: mod_base = OTHER; note = locked nucleic acid (LNA) nucleotide
catggtgtcttatgctgtcc

SEQ ID NO: 10 - DNA, 22 bp, synthetic construct
  source 1..22: mol_type = other DNA
cgctgttgaatacgcttaacat

SEQ ID NO: 11 - DNA, 29 bp, synthetic construct
  source 1..29: mol_type = other DNA
ttttggatagtaaaataagtttcgaactc

SEQ ID NO: 12 - DNA, 22 bp, synthetic construct
  source 1..22: mol_type = other DNA
  modified_base 3: mod_base = OTHER; note = locked nucleic acid (LNA) nucleotide
  modified_base 7: mod_base = OTHER; note = locked nucleic acid (LNA) nucleotide
  modified_base 16: mod_base = OTHER; note = locked nucleic acid (LNA) nucleotide
  modified_base 18: mod_base = OTHER; note = locked nucleic acid (LNA) nucleotide
acctttcaattttgtcgcactc